(19) United States
(12) Patent Application Publication
(10) Pub. No.: US 2008/0004389 A1
(43) Pub. Date: Jan. 3, 2008

(54) Polymer Composition for Air Purification

(75) Inventors: Man-Khyun Rho, Seoul (KR); Tae-Hoon Jurng, Seoul (KR)

Correspondence Address: BACON & THOMAS, PLLC, 625 SLATERS LANE, FOURTH FLOOR, ALEXANDRIA, VA, 22314, US

(73) Assignee: Man-Khyun Rho, Seoul (KR)

(21) Appl. No.: 11/667,587

(22) PCT Filed: Sep. 26, 2005
(86) PCT No.: PCT/KR05/03184

§ 371 Date: May 11, 2007

(30) Foreign Application Priority Data
Nov. 13, 2004 (KR) 10-2004-0092823

(51) Int. Cl. A61L 9/01; C08G 77/04

(52) U.S. Cl. 524/430; 524/401; 524/433; 524/436

(57) ABSTRACT

Disclosed herein are a polymer composition for air purification and a filter manufactured using the polymer composition. The polymer composition comprises a peroxide or superoxide and silicone (i.e. an organopolysiloxane). Since the silicone has a high gas permeability, the polymer composition comprising the peroxide or superoxide as an air purifier rapidly absorbs harmful gases and is highly stable even when in contact with water, allowing the polymer composition to show superior characteristics. Particularly, since the polymer composition comprises a stabilizer, it is highly stable and thus is not spontaneously ignited even at very high temperatures. Accordingly, the polymer composition can be manufactured into filters having various shapes by common rubber processing.

### TECHNICAL FIELD

The present invention relates to a composition for air purification. More particularly, the present invention relates to a polymer composition for air purification comprising a peroxide or superoxide for removing harmful gases and generating oxygen and a silicone resin, and a filter manufactured using the polymer composition.

### BACKGROUND ART

In general, filters for use in air purification systems are used to remove airborne dust particles. For this purpose, a filter known as a high-efficiency particulate air (HEPA) filter is currently used. HEPA filters are highly advantageous in the removal of dust particles. Specifically, a HEPA filter with a removal efficiency of 99% or more on house dust mites, viruses, molds, particles having a size of 0.3 μm or more, etc., are suited for use in an air purification system.

In addition to HEPA filters, a variety of functional filters, such as activated charcoal filters for removing unpleasant smells, filters for generating anions, photocatalyst-coated filters and antibacterial filters, are currently used in air purification systems.

However, these functional filters, including HEPA filters, are effective in the removal of dust particles and organic materials causing unpleasant odors, but they are not suitable for the removal of acidic, harmful gases, i.e., sulfur oxides (SOx), nitrogen oxides (NOx) and carbon dioxide (CO₂), as air pollutants.

The most general process for removing such harmful gases is to pass polluted air through a column, packed with a air purifying agent comprising a strongly alkaline material selected from lithium hydroxide (LiOH), sodium hydroxide (NaOH), calcium hydroxide (Ca(OH)₂), soda lime, etc., as an active component. However, since this column process requires a sophisticated mechanical system and a large space for installation, it is difficult to apply to a filter for an air purification system and is thus limited to special situations, such as submarines.

For convenience of use, there have been several attempts to mix the strongly alkaline air purifier with a polymer resin.

U.S. Pat. No. 5,165,399 discloses a carbon dioxide absorbent in the form of a sheet produced by admixing lithium hydroxide (LiOH) with polytetrafluoroethylene (PTFE), and a filter for an air purification system manufactured by processing the carbon dioxide absorbent. The deficiency of this technique is that additional support materials, such as woven fabrics or non-woven fabrics, must be used for structural integrity of the carbon dioxide absorbent. In particular, the processing into a thin film or a fiber or filament having a small diameter is extremely difficult.

Meanwhile, U.S. Pat. No. 5,964,221 mentions a sheet produced by mixing an alkaline air purifier, such as lithium hydroxide or calcium hydroxide, an ultra-high molecular weight polyethylene (UHMWPE) and mineral oil, and melt-extruding the mixture. Since this technique allows the sheet to have a single structure, no additional support non-woven fabric or woven fabric is needed to maintain structural integrity, unlike in U.S. Pat. No. 5,165,399.

However, the use of mineral oil as a lubricant during extrusion involves a troublesome subsequent step of washing the mineral oil with an organic solvent. Particularly, since the mineral oil and the organic solvent are highly flammable, this technique cannot be applied to highly oxidative air purifiers, such as potassium superoxide and sodium peroxide.

Potassium superoxide and sodium peroxide are used as air revitalization materials or air purifiers because they can fix carbon dioxide present in air and generate oxygen as depicted in Reactions 1 and 2 below:
2KO₂+CO₂→K₂CO₃+3/2O₂ ChemistryFigure 1
Na₂O₂+CO₂→Na₂CO₃+1/2O₂ ChemistryFigure 2

In addition to these compounds, potassium peroxide (K₂O₂), calcium peroxide (CaO), lithium peroxide (Li₂O₂), and sodium superoxide (NaO₂) absorb carbon dioxide and generate oxygen contained in air, and thus can be used as air purifiers.

The above-mentioned peroxides and superoxides are produced in the form of pellets and filled into a container, e.g., a column, for an air purification system. A small amount of a binder is commonly added to improve the mechanical properties of the pellets and to prevent the pellets from being fused.

For example, PCT Publication WO 03/009899 A1 describes a composition for air purification in which at most 3.0 wt % of a binder is mixed with an alkali metal or alkaline earth metal superoxide or peroxide. The binder is selected from inorganic binders, e.g., sodium silicate (Na₂SiO₃) and potassium silicate (K₂SiO₃); and organic binders, e.g., sodium polyvinyl tetrazole, sodium carboxyl cellulose, polyvinyl acetate, nitro cellulose, and epoxy resins.

In an effort to solve the fusion problem of potassium superoxide and further to improve the oxygen generation performance, U.S. Pat. No. 4,113,646 discloses the addition of anhydrous calcium sulfate (CaSO₄), silicon dioxide (SiO₂), lithium monoxide (Li₂O), lithium metaborate (LiBO₂), and the like.

U.S. Pat. No. 4,238,464 discloses a composition for reducing the fusion of potassium superoxide, which comprises a salt containing at least one element selected from zirconium, titanium and boron, and potassium superoxide.

In particular, severe fusion of potassium superoxide inside a bed filled with potassium superoxide creates a considerable pressure drop of air passing through the bed. U.S. Pat. No. 4,490,272 discloses the addition of 2˜30 wt % of an alkaline earth metal oxide, such as CaO, in order to remarkably improve the problem of pressure drop resulting from heat fusion.

Upon reviewing these technologies hitherto reported, the present inventors found that the materials added to peroxides or superoxides are a kind of binder for maintaining the geometric shape of pellets produced from the mixtures.

### DISCLOSURE OF INVENTION

### Technical Problem

It is a first object of the present invention to provide a polymer composition for air purification comprising a peroxide or superoxide and silicone.

Since silicone has much higher gas permeability than any other polymer resin, harmful gases can easily pass through the silicone at a high rate. Particular, since silicone is highly water repellent, a combination with a peroxide or superoxide does not readily react with water when in direct contact with water.

Since superoxides and peroxides have a strong oxidizing power, they may be spontaneously ignited when being blended with a molten polymer. In contrast, since silicone is cured at room temperature, a blend with a superoxide or peroxide offer little or no danger of spontaneous ignition.

Accordingly, the composition comprising a silicone resin and a peroxide or superoxide according to the first object serves to overcome the above-mentioned problems of conventional compositions for air purification in the form of pellets.

It is a second object of the present invention to provide a polymer composition for air purification with improved flame retardance, comprising a peroxide or superoxide and a component for stabilizing the strong oxidizing power of the peroxide or superoxide, the stabilizer being selected from hydroxides, e.g., calcium hydroxide (Ca(OH)₂), aluminum hydroxide (Al(OH)₃), magnesium hydroxide (Mg(OH)₂), barium hydroxide (Ba(OH)₂), lithium hydroxide (LiOH), sodium hydroxide (NaOH) and potassium hydroxide (KOH) and flame retardants.

It is a third object of the present invention to provide a filter for absorbing acidic gases, e.g., sulfur oxides (SOx), nitrogen oxides (NOx), carbon dioxide (CO₂) etc., and generating oxygen, the filter being manufactured by processing the silicone composition comprising the stabilizer and the peroxide or superoxide into a certain shape, such as a sheet, film, strip, filament, fiber, or hollow fiber.

### Technical Solution

In order to accomplish the above objects of the present invention, there is provided a polymer composition for air purification, comprising:

(A) an organopolysiloxane;

(B) a curing agent; and

(C) an air purifier.

The polymer composition of the present invention may further comprise (D) a stabilizer.

The organopolysiloxane (A) used in the present invention is the main component of the composition according to the present invention, and refers to a compound containing a silicon-oxygen bond (Si—O) as a repeating unit of the backbone. The organopolysiloxane is represented by the average formula [RₘSiO_{(4-m)/2}], has a liquid, resin or elastomer shape, and may be of a linear, branched, partially branched linear, dendritic molecular structure.

In the above formula, the substituents R may be selected from: alkyl groups, e.g., methyl, ethyl, propyl, butyl, and octyl; aryl groups, e.g., phenyl and tolyl; aralkyl groups, e.g., benzyl and phenethyl; cycloalkyl groups, e.g., cyclopentyl and cyclohexyl; alkenyl groups, e.g., vinyl, ally, butenyl, hexenyl, and heptenyl; halogenated alkyl groups, e.g., 3,3,3-trifluoropropyl and chloropropyl. Among these groups, alkyl, alkenyl and aryl groups are preferred, and methyl, vinyl and phenyl groups are more preferred. The letter “m” is a positive number of 1.8 to 2.3.

Data regarding the chemical and physical properties and the kinds of the organopolysiloxanes can be found in the literature, for example, “Encyclopedia of polymer science and engineering”, volume 15, pp. 204-308, Wiley-Interscience (1989).

The organopolysiloxanes can be crosslinked (i.e. cured) by the action of curing agents. The organopolysiloxanes are cured via a hydrosilylation reaction, a condensation reaction or a free-radical reaction depending on their chemical structure.

Organopolysiloxanes cured via a hydrosilylation reaction include those having, on average, not less than 0.1 silicon-bonded alkenyl groups per molecule, preferably, on average, not less than 0.5 silicon-bonded alkenyl groups per molecule, and especially preferably, on average, not less than 0.8 silicon-bonded alkenyl groups per molecule.

When the average number of silicon-bonded alkenyl groups per molecule is lower than the lower limit of the above-mentioned range, the resultant composition may be not completely cured. Examples of silicon-bonded alkenyl groups include vinyl, allyl, butenyl, pentenyl, and hexenyl, of which vinyl is preferred.

Specific examples of organopolysiloxanes suitable for use in the hydrosilylation include a dimethylpolysiloxane having both terminal ends of the molecular chain blocked by dimethylvinylsiloxy groups, a dimethylpolysiloxane having both terminal ends of the molecular chain blocked by methylphenylvinylsiloxy groups, a methylphenylsiloxane-dimethylsiloxane copolymer having both terminal ends of the molecular chain blocked by dimethylvinylsiloxy groups, a methylvinylsiloxane-dimethylsiloxane copolymer having both terminal ends of the molecular chain blocked by dimethylvinylsiloxy groups, a methylvinylsiloxane-dimethylsiloxane copolymer having both terminal ends of the molecular chain blocked by trimethylsiloxy groups, a methyl(3,3,3-trifluoropropyl)polysiloxane having both terminal ends of the molecular chain blocked by dimethylvinylsiloxy groups, a methylvinylsiloxane-dimethylsiloxane copolymer having both terminal ends of the molecular chain blocked by silanol groups, a methylphenylsiloxane-methylvinylsiloxane-dimethylsiloxane copolymer having both terminal ends of the molecular chain blocked by silanol groups, and an organosiloxane copolymer made up of siloxane units represented by the formula (CH₃)₃SiO_{1/2}, siloxane units represented by the formula (CH₃)₂(CH₂═CH)SiO_{1/2}, siloxane units represented by the formula CH₃SiO_{3/2}, and siloxane units represented by the formula (CH₃)₂SiO_{2/2}, and mixtures thereof.

When the organopolysiloxane (A) is cured via a hydrosilylation reaction, the curing agent is preferably used in an amount of 0.0001˜0.5 parts by weight, and particularly 0.001˜0.4 parts by weight, based on one part by weight of the organopolysiloxane (A) as the main component.

When the organopolysiloxane (A) is cured via a condensation reaction, the organopolysiloxane has at least two silanol groups or silicon-bonded hydrolyzable groups in one molecule.

Examples of silicon-bonded hydrolyzable groups include methoxy, ethoxy, propoxy, and other alkoxy groups; vinyloxy and other alkenoxy groups; methoxyethoxy, ethoxyethoxy, methoxypropoxy and other alkoxyalkoxy groups; acetoxy, octanoyloxy and other acyloxy groups; dimethylketoxime, methylethylketoxime, and other ketoxime groups; isopropenyloxy, 1-ethyl-2-methylvinyloxy and other alkenyloxy groups; dimethylamino, diethylamino, butylamino, and other amino groups; dimethylaminoxy, diethylaminoxy, and other aminoxy groups; N-methylacetamide, N-ethylacetamide, and other amide groups.

Examples of groups in the organopolysiloxane other than the silanol or silicon-bonded hydrolyzable groups include methyl, ethyl, propyl, and other alkyl groups; cyclopentyl, cyclohexyl, and other cycloalkyl groups; vinyl, allyl, and other alkenyl groups; phenyl, naphthyl, and other aryl groups; and 2-phenylethyl and other aralkyl groups.

When the organopolysiloxane (A) is cured via a free-radical reaction, it preferably has at least one silicon-bonded alkenyl group per molecule. Examples of silicon atom-bonded alkenyl groups in the organopolysiloxane include vinyl, allyl, butenyl, pentenyl, and hexenyl groups. Vinyl is particularly preferred.

In addition to these alkenyl groups, examples of silicon atom-bonded groups in the organopolysiloxane include methyl, ethyl, propyl, butyl, pentyl, hexyl, and other alkyl groups; cyclopentyl, cyclohexyl, and other cycloalkyl groups; phenyl, tolyl, xylyl, and other aryl groups; benzyl, phenethyl, and other aralkyl groups; and 3,3,3-trifluoropropyl, 3-chloropropyl, and other halogenated alkyl groups. Of these groups, alkyl and aryl groups are preferred, and methyl and phenyl are particularly preferred.

When the organopolysiloxane (A) is cured via a hydrosilylation reaction, the curing agent (B) consists of a platinum catalyst and an organopolysiloxane having, on average, not less than two silicon-bonded hydrogen atoms per molecule.

Examples of silicon-bonded groups contained in the organopolysiloxane include methyl, ethyl, propyl, butyl, pentyl, hexyl, and other alkyl groups; cyclopentyl, cyclohexyl, and other cycloalkyl groups; phenyl, tolyl, xylyl, and other aryl groups; benzyl, phenethyl, and other aralkyl groups; and 3,3,3-trifluoropropyl, 3-chloropropyl, and other halogenated alkyl groups. Of the preceding groups, alkyl and aryl groups are preferred, and methyl and phenyl are particularly preferred.

The platinum catalyst is a catalyst used for promoting the curing of the composition of the present invention. Chloroplatinic acid, alcohol solutions of chloroplatinic acid, olefin complexes of platinum, alkenylsiloxane complexes of platinum, and platinum carbonyl complexes are suggested as examples thereof.

In the present composition, the content of the platinum catalyst is such that the amount of the platinum metal is within the range of from 0.2 ppm to 200 ppm, and preferably, within the range of from 0.1 ppm to 500 ppm, based on the weight of the organopolysiloxane (A) as the main component. When the content of the platinum catalyst is lower than the lower limit of the above-mentioned range, the resultant composition for air purification is not cured completely, and, on the other hand, adding an amount exceeding the upper limit of the above-mentioned range does not lead to an increase in the curing rate of the resultant composition for air purification.

When the organopolysiloxane (A) is cured via a condensation reaction, the curing agent (B) is a silane having at least three silicon-bonded groups hydrolyzable groups in one molecule, a hydrolysis product thereof, and, if necessary, a condensation reaction catalyst.

Examples of silicon-bonded hydrolyzable groups include methoxy, ethoxy, propoxy, and other alkoxy groups; vinyloxy and other alkeneoxy groups; methoxyethoxy, ethoxyethoxy, methoxypropoxy and other alkoxyalkoxy groups; acetoxy, octanoyloxy and other acyloxy groups; dimethylketoxime, methylethylketoxime, and other ketoxime groups; isopropenyloxy, 1-ethyl-2-methylvinyloxy and other alkenyloxy groups; dimethylamino, diethylamino, butylamino, and other amino groups; dimethylaminoxy, diethylaminoxy, and other aminoxy groups; N-methylacetamide, and N-ethylacetamide.

In addition, hydrocarbon groups may be bonded to the silane. Examples of such hydrocarbon groups include methyl, ethyl, propyl, butyl, pentyl, hexyl, heptyl, octyl, nonyl, decyl, octadecyl, and other alkyl groups; cyclopentyl, cyclohexyl, and other cycloalkyl groups; vinyl, allyl, and other alkenyl groups; phenyl, tolyl, xylyl, naphthyl, and other aryl groups; benzyl, phenethyl, phenylpropyl and other aralkyl groups, and 3-chloropropyl, 3,3,3-trifluoropropyl and other halogenated alkyl groups.

Methyltriethoxysilane, vinyltriethoxysilane, vinyltriacetoxysilane, and ethyl or thosilicate are suggested as examples of the silane or its partial hydrolysis products.

In the present composition, the content of the silane or its partial hydrolysis products is preferably within the range of from 0.0001 parts by weight to 0.5 parts by weight, and especially preferably within the range of from 0.001 parts by weight to 0.4 parts by weight, per one part by weight of the organopolysiloxane as the main component. When the content of the silane or its partial hydrolysis product is lower than the lower limit of the above-mentioned range, the storage stability of the resultant composition tends to deteriorate and its adhesion properties tend to decrease, and, on the other hand, when the content exceeds the upper limit of the above-mentioned range, the curing rate of the resultant composition tends to considerably slow down.

Examples of condensation reaction catalysts that can be used in the composition of the present invention include tetrabutyl titanate, tetraisopropyl titanate, and other titanic acid esters; diisopropoxybis(acetylacetato)titanium, diisopropoxybis(ethylacetoacetato)titanium, and other chelated organotitanium compounds; aluminum tris(acetylacetonate), aluminum tris(ethylacetoacetate), and other organoaluminum compounds; zirconium tetra(acetylacetotonate), zirconium tetrabutylate, and other organozirconium compounds; dibutyltin dioctoate, dibutyltin dilaurate, butyltin-2-etylhexoate, and other organotin compounds; tin naphthenoate, tin oleate, tin butylate, cobalt naphthenoate, zinc stearate, and other metal salts of organic carboxylic acids; hexylamine, dodecylamine phosphate, and other amine compounds and their salts; benzyltriethylammonium acetate, and other quaternary ammonium salts; lower fatty acid salts of alkali metals such as lithium nitrate and potassium acetate; dimethylhydroxylamine, diethylhydroxylamine, and other dialkylhydroxylamines; and guanidyl group-containing organosilicon compounds, and the like.

The content of the condensation reaction catalyst used in the present invention is preferably within the range of from 0.0001 parts by weight to 0.2 parts by weight, and, especially preferably, within the range of from 0.001 parts by weight to 0.1 parts by weight, per one part by weight of the organopolysiloxane (A) as the main component. When this catalyst is necessary, if the content of the catalyst is lower than the lower limit of the above-mentioned range, the resultant composition often is not cured completely, and, on the other hand, when it exceeds the upper limit of the above-mentioned range, the storage stability of the resultant composition tends to deteriorate.

In addition, when the curing reaction is a free-radical reaction, the curing agent (B) is an organic peroxide. Examples of such organic peroxides include benzoyl peroxide, dicumyl peroxide, 2,5-dimethylbis(2,5-t-butylperoxy)hexane, di-tert-butyl peroxide, and tert-butyl perbenzoate. The amount of the organic peroxides added is within the range of from 0.001 parts by weight to 0.05 parts by weight, per one part by weight of the organopolysiloxane (A).

The kinds of the organopolysiloxane (A), the curing agent (B), and the curing catalyst are well known to those skilled in the art, and can found in, for example, U.S. Pat. Nos. 6,380,301 and 6,387,971, and Korean Patent Laid-open No. 2003-0097869.

The air purifier (C) constituting the polymer composition for air purification according to the present invention is selected from peroxides and superoxides, e.g., sodium peroxide (Na₂O₂), potassium peroxide (K₂O₂), calcium peroxide (CaO₂), lithium peroxide (Li₂O₂), sodium superoxide (NaO₂) and potassium superoxide (KO₂), and mixtures thereof. Since these materials can absorb acidic, harmful gases, such as SOx, NOx and CO₂, and can generate oxygen, they can be used as air revitalization materials.

In addition to the peroxide or superoxide, there are a number of oxygen-generating materials, for example, alkali metal chlorates and perchlorates, including lithium perchlorate (LiClO₄), lithium chlorate (LiClO₃), sodium perchlorate (NaClO₄), sodium chlorate (NaClO₃), sodium perchlorate (KClO₄), and sodium chlorate (KClO₃). However, since these chlorates or perchlorates generate oxygen as a decomposition product when being heated by electrical or chemical techniques, they are poor in the absorption of harmful gases as compared to the above-mentioned peroxides and superoxides.

On the other hand, representative air purifiers capable of removing carbon dioxide in air are soda lime, which is a mixture of calcium hydroxide (Ca(OH)₂) and sodium hydroxide, and strongly alkaline materials, such as lithium hydroxide. However, since these air purifiers remove acidic gases but do not generate oxygen, they are disadvantageous over sodium peroxide and potassium superoxide in terms of air purification efficiency.

Accordingly, sodium peroxide and potassium superoxide having superior oxygen generation ability are advantageously used in self-contained breathing apparatuses, as compared with the use of carbon dioxide absorbers.

The alkali metal or alkaline earth metal peroxide or superoxide is preferably used in an amount of 0.01 to 98 parts by weight and more preferably in an amount of 0.05 to 20 parts by weight, based on one part by weight of the organopolysiloxane (A). When the content of the superoxide or peroxide is lower than the lower limit of the above-mentioned range, the air purification efficiency of the superoxide or peroxide is meaningless. Meanwhile, when the content of the peroxide or superoxide exceeds the upper limit of the above-mentioned range, the constituent components of the composition do not tend to be homogeneously distributed in a matrix of the silicone polymer.

The alkali or alkaline earth metal peroxide or superoxide most preferably has a powdery form to allow easy mixing with the organopolysiloxane (A).

The stabilizer (D) is used to inhibit the strong oxidizing power of the peroxide or superoxide in the polymer composition for air purification. This is because a mixture of the organopolysiloxane with the peroxide or superoxide (C) is highly combustible. The use of the stabilizer (D) prevents the composition from being highly flammable

Examples of suitable stabilizers that can be used in the present invention include hydroxides, such as calcium hydroxide (Ca(OH)₂), aluminum hydroxide (Al(OH)₃), magnesium hydroxide (Mg(OH)₂), barium hydroxide (Ba(OH)₂), lithium hydroxide (LiOH), sodium hydroxide (NaOH) and potassium hydroxide (KOH).

Mixing of the peroxide or superoxide with the hydroxide reduces the oxidizing power of the superoxide or peroxide to any appreciable extent, and prevents inflammation of the composition according to the present invention. The high or low alkalinity of the hydroxide assists in absorbing acidic gases. Accordingly, the hydroxide is advantageous over common polymer fillers, and thus lithium hydroxide, sodium hydroxide, potassium hydroxide or the like is usable not only as the stabilizer (D) but also as the air purifier (C). The amount of the hydroxide as the air purifier (D) used is preferably between 0.01 and 98 parts by weight, and more preferably between 0.05 and 20 parts by weight, based on one part by weight of the organopolysiloxane (A), similarly to that of the peroxide or superoxide.

A common inorganic filler for polymer processing can be used as the stabilizer, and examples of such inorganic fillers include clay, calcium carbonate (CaCO₃), talc, diatomaceous earth, carbon black, silica, and glass fiber.

A flame retardant for polymer processing can be used as the stabilizer, and examples of such flame retardants are inorganic flame retardants, including antimony compounds, such as antimony trioxide, antimony pentoxide and sodium antimonate; boron compounds, such as boric acid, borax and zinc borate; molybdenum compounds; titanium compounds; zirconium compounds; tin compounds, such as zinc stannate; phosphorus compounds, such as red phosphorus and ammonium polyphosphate; ammonium sulfamate; and ammonium bromide.

Further, a halogenated organic compound can be used as the stabilizer, and examples of suitable halogenated organic compounds include tetrabromobisphenol A, decabromodiphenyl ether, octabromobiphenyl ether, tetrabromobiphenyl ether, hexabromocyclododecane, tribromophenol, bis(tribromophenoxy) ethane, tetrabromobisphenol A polycarbonate oligomers, tetrabromobisphenol A epoxy oligomers, chlorinated paraffins, and bis(hexachlorocyclopentadieno)cyclooctane.

An organophosphorus compound can be used as the stabilizer, and examples of suitable organophosphorus compounds include trialkyl phosphates, triaryl phosphates, aryl-alkyl phosphates, phosphonium derivatives, phosphonates, tris(1-chloro-2-propyl) phosphate, tris(2-chloroethyl) phosphate, and tris(2,3-dibromopropyl)phosphate.

In addition to these stabilizers, a nitrogen compound can be used as the stabilizer. Examples of suitable nitrogen compounds include melamine and its salts, such as melamine cyanurate and melamine polyphosphate, and guanidine compounds.

When the stabilizer is an inorganic material, it is preferred that the stabilizer is first mixed with the peroxide or superoxide and then the mixture is mixed with the other components. Meanwhile, when the stabilizer is an organic material, it is preferred that the stabilizer is first mixed with the organopolysiloxane and then the mixture is mixed with the other components. But, the mixing order of the components is not particularly limited.

The above-mentioned stabilizers may be used alone or in combination as a mixture of two or more of the stabilizers to exert a synergic effect.

Among these stabilizers, the hydroxide or the filler for polymer processing is preferably used in an amount of 0.05 to 20 parts by weight, based on one part by weight of the peroxide or superoxide. If the amount of the hydroxide or the filler is lower than the lower limit of the defined range, the oxygen generation is poor and air purification efficiency is low. On the other hand, when the amount exceeds the upper limit, the reduction in the oxidizing power of the peroxide or superoxide is hard to control.

The flame retardant for polymer processing has strong stabilization effects on the peroxide or superoxide when compared to common hydroxides and fillers. Accordingly, even when the flame retardant is used in a small amount, desired effects, i.e. flammability prevention, can be achieved. The flame retardant for polymer processing is preferably used in an amount of 0.01 to 10 parts by weight, based on one part by weight of the peroxide or superoxide.

The amount of the stabilizer used varies depending on the desired application of the polymer composition for air purification according to the present invention. Namely, when the polymer composition for air purification according to the present invention is intended to be used in a closed-circuit rebreather device where strong air purification effects are required, the stabilizer should be used in an amount as small as possible or its use should be avoided.

Any additional components, such as foaming agents, plasticizers, pigments, dyes, fluorescent dyes and heat resistant additives, can be added to the polymer composition for air purification of the present invention so far as they do not impair the object of the present invention.

Further, when the composition of the present invention is cured via a hydrosilylation reaction, a curing reaction inhibitor may be added to the composition of the present invention to control the curing rate and improve the handling workability of the composition. Examples of suitable curing reaction inhibitors include acetylene compounds, such as 2-methyl-3-butyn-2-ol, 2-phenyl-3-butyn-2-ol and 1-ethynyl-1-cyclohexanol; ene-yne compounds, such as 3-methyl-3-penten-1-yne and 3,5-dimethyl-3-hexene-1-yne; hydrazine compounds; and phosphine compounds.

The curing reaction of the polymer composition for air purification according to the present invention can be performed at room temperature or high temperature, depending on the reaction mechanisms. Generally, the polymer composition of the present invention is molded into various shapes, such as sheets, films, filaments, fibers and hollow fibers, using compounders and molding devices commonly used in the rubber industry.

The molded polymer composition of the present invention does not readily react with water, despite being in direct contact with water due to the presence of the peroxide or superoxide inside a matrix of the silicone rubber.

Since the silicone rubber has much higher gas permeability than common polymers, such as vinyl, polyester and polyamide resins, acidic gases present in air can easily pass through the silicon rubber at a high rate. Therefore, the molded polymer composition of the present invention can be feasibly applied to filters for removing harmful gases.

### Advantageous Effects

The polymer composition for air purification according to the present invention provides several advantages as follows:

Firstly, the polymer composition of the present invention is highly stable even when in contact with water. In contrast, common peroxides and superoxides vigorously react with water.

Secondly, since the silicone used in the present invention has a high gas permeability, the peroxide or superoxide present in a silicone matrix rapidly absorbs harmful gases present in air, allowing the polymer composition of the present invention to show superior air purification performance.

Thirdly, since the polymer composition according to the present invention comprises a stabilizer, it is highly stable and thus is not spontaneously ignited even at very high temperatures. Accordingly, the polymer composition of the present invention can be manufactured into filters having various shapes by common rubber processing.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### BRIEF DESCRIPTION OF THE DRAWINGS

The above and other objects, features and other advantages of the present invention will be more clearly understood from the following detailed description taken in conjunction with the accompanying drawing, in which:

FIG. 1 is a graph comparing the carbon dioxide absorption rate of polymer compositions (Sample Nos. 1-4) for air purification according to the present invention with that of pure potassium superoxide (KO₂) and sodium hydroxide (NaOH) pellet.

### BEST MODE FOR CARRYING OUT THE INVENTION

The present invention will now be described in more detail with reference to the following examples.

### EXAMPLE 1

Dimethylpolysiloxane with a viscosity of 100 mPa [symbol] s at room temperature having both terminal ends of the molecular chain blocked by hydroxyl groups, methyltriacetoxysilane, dibutyltin dilaurate, and potassium superoxide were mixed in accordance with the respective contents indicated in Table 1 below, to prepare polymer compositions (Sample Nos. 1-4) for air purification. The polymer compositions were sufficiently cured and stored in a desiccator filled with nitrogen.

TABLE 1
Components and contents of each sample for air purification (Example 1)

| | Dimethylpolysiloxane | | Dibutyltin | Potassium |
|---|---|---|---|---|
| Sample | (Parts by | Methyltriacetoxysilane | dilaurate(Parts | superoxide(Parts by |
| No. | weight) | (Parts by weight) | by weight) | weight) |
| 1 | 45.42 | 4.54 | 0.0454 | 50 |
| 2 | 54.50 | 5.49 | 0.0545 | 40 |
| 3 | 63.58 | 6.36 | 0.0636 | 30 |
| 4 | 72.66 | 7.26 | 0.0726 | 20 |

The color of the polymer compositions (Sample Nos. 1-4) was light yellow, and turned white after the potassium superoxide completely reacted with carbon dioxide to generate oxygen.

### EXAMPLE 2

Dimethylpolysiloxane with a viscosity of 100 mPa [symbol] s at room temperature having both terminal ends of the molecular chain blocked by hydroxyl groups, methyltriacetoxysilane, dibutyltin dilaurate, potassium superoxide, and calcium hydroxide were mixed in accordance with the respective contents indicated in Table 2 below, to prepare polymer compositions (Sample Nos. 5-8) for air purification. The polymer compositions were sufficiently cured and stored in a desiccator filled with nitrogen.

TABLE 2
Components and contents of each sample for air purification (Example 2)

| | | | Dibutyltin | Potassium | |
|---|---|---|---|---|---|
| | Dimethylpolysiloxane | Methyltriacetoxysilane | dilaurate (Parts | superoxide (Parts | Calcium |
| Sample | (Parts | (Parts | by | by | hydroxide (Parts by |
| No. | by weight) | by weight) | weight) | weight) | weight) |
| 5 | 45.42 | 4.54 | 0.0454 | 40 | 10 |
| 6 | 45.42 | 4.54 | 0.0454 | 30 | 20 |
| 7 | 45.42 | 4.54 | 0.0454 | 20 | 30 |
| 8 | 45.42 | 4.54 | 0.0454 | 10 | 40 |

### EXAMPLE 3

Dimethylpolysiloxane with a viscosity of 100 mPa [symbol] s at room temperature having both terminal ends of the molecular chain blocked by hydroxyl groups, methyltriacetoxysilane, dibutyltin dilaurate, potassium superoxide, and magnesium hydroxide were mixed in accordance with the respective contents indicated in Table 3 below, to prepare polymer compositions (Sample Nos. 9-12) for air purification. The polymer compositions were sufficiently cured and stored in a desiccator filled with nitrogen.

TABLE 3
Components and contents of each sample for air purification (Example 3)

| | | | Dibutyltin | Potassium | Magnesium |
|---|---|---|---|---|---|
| | Dimethylpolysiloxane | | dilaurate | superoxide | hydroxide |
| Sample | (Parts by | Methyltriacetoxysilane | (Parts by | (Parts by | (Parts by |
| No. | weight) | (Parts by weight) | weight) | weight) | weight) |
| 9 | 45.42 | 4.54 | 0.0454 | 40 | 10 |
| 10 | 45.42 | 4.54 | 0.0454 | 30 | 20 |
| 11 | 45.42 | 4.54 | 0.0454 | 20 | 30 |
| 12 | 45.42 | 4.54 | 0.0454 | 10 | 40 |

The color of the polymer compositions prepared in Examples 2 and 3 (containing each stabilizer) turned cloudy from the light yellow color of the polymer composition prepared in Example 1 (comprising no stabilizer).

### EXAMPLE 4

Dimethylpolysiloxane with a viscosity of 100 mPa [symbol] s at room temperature having both terminal ends of the molecular chain blocked by hydroxyl groups, methyltriacetoxysilane, dibutyltin dilaurate, potassium superoxide, and fumed silica were mixed in accordance with the respective contents indicated in Table 4 below, to prepare polymer compositions (Sample Nos. 13-16) for air purification. The polymer compositions were sufficiently cured and stored in a desiccator filled with nitrogen.

TABLE 4
Components and contents of each sample for air purification (Example 4)

| | | | Dibutyltin | Potassium | |
|---|---|---|---|---|---|
| | Dimethylpolysiloxane | Methyltriacetoxysilane | dilaurate (Parts | superoxide | Fumed |
| Sample | (Parts by | (Parts by | by | (Parts by | silica (Parts by |
| No. | weight) | weight) | weight) | weight) | weight) |
| 13 | 45.42 | 4.54 | 0.0454 | 40 | 10 |
| 14 | 45.42 | 4.54 | 0.0454 | 30 | 20 |
| 15 | 45.42 | 4.54 | 0.0454 | 20 | 30 |
| 16 | 45.42 | 4.54 | 0.0454 | 10 | 40 |

### EXAMPLE 5

Dimethylpolysiloxane with a viscosity of 100 mPa [symbol] s at room temperature having both terminal ends of the molecular chain blocked by hydroxyl groups, methyltriacetoxysilane, dibutyltin dilaurate, potassium superoxide, and melamine cyanurate were mixed in accordance with the respective contents indicated in Table 5 below, to prepare polymer compositions (Sample Nos. 17-20) for air purification. The polymer compositions were sufficiently cured and stored in a desiccator filled with nitrogen.

TABLE 5
Components and contents of each sample for air purification (Example 5)

| | | | Dibutyltin | Potassium | |
|---|---|---|---|---|---|
| | Dimethylpolysiloxane | Methyltriacetoxysilane | dilaurate | superoxide | Melaminecyanurate |
| Sample | (Parts | (Parts by | (Parts by | (Parts by | (Parts |
| No. | by weight) | weight) | weight) | weight) | by weight) |
| 17 | 45.42 | 4.54 | 0.0454 | 40 | 10 |
| 18 | 45.42 | 4.54 | 0.0454 | 30 | 20 |
| 19 | 45.42 | 4.54 | 0.0454 | 20 | 30 |
| 20 | 45.42 | 4.54 | 0.0454 | 10 | 40 |

### EXAMPLE 6

### Comparison of Carbon Dioxide Absorption

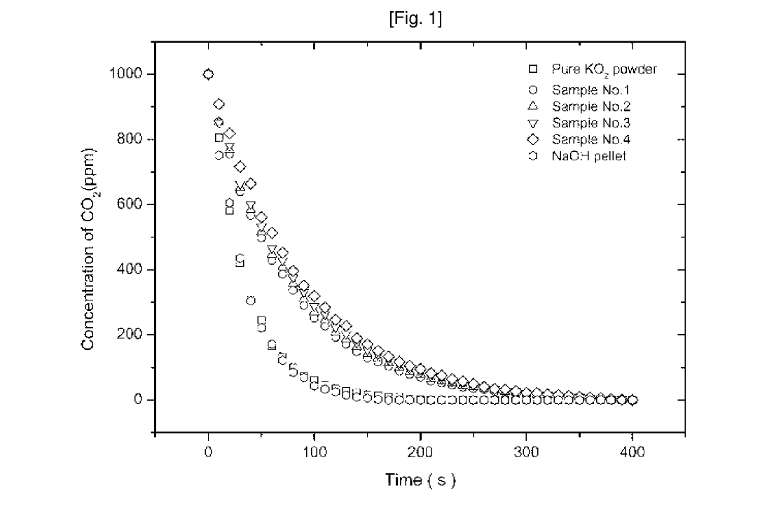

FIG. 1 is a graph comparing the carbon dioxide absorption rate of the polymer compositions for air purification (Sample Nos. 1-4), pure potassium superoxide, and NaOH.

All experiments were conducted in a cylindrical container having an internal volume of 2 L. A pure potassium superoxide powder, a NaOH pellet, and the samples prepared in Example 1 were placed in the container. Carbon dioxide was charged into the container using a syringe until the initial concentration of carbon dioxide reached 1,000 ppm, and then the changes in the concentration of the carbon dioxide were recorded.

At this time, the polymer compositions were in the form of a sphere having an average diameter of 3 mm, the pure potassium superoxide was in the form of a powder, and the NaOH was in the form of a pellet having a diameter of 0.5 mm, each of which was placed on the bottom of the container.

As apparent from the graph shown in FIG. 1, it has been confirmed that the polymer compositions had a relatively low carbon dioxide absorption rate compared to the pure potassium superoxide. The reason for this is believed to be that the polymer compositions for air purification have a resistance against the diffusion of carbon dioxide to some extent due to the presence of the CO₂-absorbing potassium superoxide inside the matrix.

Despite the resistance against gas diffusion, there was no problem in removing harmful gases present in air using the polymer compositions for air purification. This is because the polymer compositions increase the contact area with air so that larger mounts of harmful gases can be removed per unit time.

Particularly, since the polymer compositions for air purification show processability comparable to common polymers, they are readily processed into various shapes having a larger contact area than pellets of inorganic materials.

### EXAMPLE 7

### Measurement of Spontaneous Ignition Temperature According to the Kind and Content of Stabilizers

In this example, the spontaneous ignition temperature of each sample prepared in Examples 1 to 5 was measured. The spontaneous ignition temperature was measured by introducing 2 g of each sample into a ceramic crucible, followed by heating in an electric oven to a given temperature. The obtained results are listed in Table 6 below.

TABLE 6
Spontaneous ignition temperature of polymer compositions for
air purification according to stabilizer

| | Content of | | |
|---|---|---|---|
| | KO₂ in | | |
| | total weight | | |
| Sample | of composition | | Spontaneous ignition |
| No. | (wt %) | Stabilizer | temperature (° C.) |
| 1 | 50 | — | 114 |
| 2 | 40 | — | 116 |
| 3 | 30 | — | 118 |
| 4 | 20 | — | 120 |
| 5 | 40 | Ca(OH)₂ 10 wt % | No spontaneous |
| | | | ignition |
| 6 | 30 | Ca(OH)₂ 20 wt % | |
| 7 | 20 | Ca(OH)₂ 30 wt % | |
| 8 | 10 | Ca(OH)₂ 40 wt % | |
| 9 | 40 | Mg(OH)₂ 10 wt % | |
| 10 | 30 | Mg(OH)₂ 20 wt % | |
| 11 | 20 | Mg(OH)₂ 30 wt % | |
| 12 | 10 | Mg(OH)₂ 40 wt % | |
| 13 | 40 | Fumed silica 10 wt % | |
| 14 | 30 | Fumed silica 20 wt % | |
| 15 | 20 | Fumed silica 30 wt % | |
| 16 | 10 | Fumed silica 40 wt % | |
| 17 | 40 | Melamine cyanurate | |
| | | 10 wt % | |
| 18 | 30 | Melamine cyanurate | |
| | | 20 wt % | |
| 19 | 20 | Melamine cyanurate | |
| | | 30 wt % | |
| 20 | 10 | Melamine cyanurate | |
| | | 40 wt % | |

As can be seen from the data shown in Table 6, the samples comprising no stabilizer were easily ignited at spontaneous ignition temperatures of 114˜133° C.

In contrast, the samples comprising the respective stabilizers, such as calcium hydroxide, were not spontaneously ignited, and instead the silicone contained in the samples was decomposed above 200° C. or higher and left behind white residues.

The inhibited spontaneous ignition provides the advantage that the compositions comprising silicone and potassium superoxide can be cured at a high temperature.

According to a common method for producing a silicone rubber, silicone gum and a curing agent are compounded at a high temperature, extruded, and calendered. Accordingly, pure potassium superoxide is difficult to use because it causes spontaneous ignition at high temperature.

However, according to the polymer composition of the present invention, the use of the stabilizer enables the processing of a silicone rubber composition comprising potassium superoxide into various shapes, e.g., films, strips, filaments, fibers and hollow fibers, by conventional rubber processing. Accordingly, filters capable of absorbing acidic gases, including sulfur oxides (SOₓ), nitrogen oxides (NOₓ) and carbon dioxide (CO₂), and generating oxygen, can be manufactured in a simple manner.

### INDUSTRIAL APPLICABILITY

As apparent from the above description, since the polymer compositions according to the present invention function to absorb carbon dioxide, carbon monoxide, SOx and NOx and transform them into oxygen, they can be widely used as a various air purifier.

1. A polymer composition for air purification, comprising:
(A) an organopolysiloxane;
(B) a curing agent; and
(C) an air purifier.

2. The polymer composition according to claim 1, wherein the air purifier (C) is selected from sodium peroxide (Na₂O₂), potassium peroxide (K₂O₂), calcium peroxide (CaO₂), lithium peroxide (Li₂O₂), sodium superoxide (NaO₂), and potassium superoxide (KO₂), and mixtures thereof; and is used in an amount of 0.05 to 20 parts by weight, based on one part by weight of the organopolysiloxane (A).

3. The polymer composition according to claim 1, wherein the air purifier (C) is selected from sodium hydroxide (NaOH), lithium hydroxide (LiOH), calcium hydroxide (Ca(OH)₂), potassium hydroxide (KOH), and mixtures thereof; and is used in an amount of between 0.05 and 20 parts by weight, based on one part by weight of the organopolysiloxane (A).

4. The polymer composition according to claim 1, further comprising a stabilizer (D) for stabilizing the oxidizing power of the air purifier.

5. The polymer composition according to claim 4, wherein the stabilizer (D) is selected from hydroxides, fillers for polymer processing, flame retardants for polymer processing, and mixtures thereof.

6. The polymer composition according to claim 5, wherein the hydroxide is used in an amount of 0.05 to 20 parts by weight, based on one part by weight of the air purifier.

7. The polymer composition according to claim 5, wherein the filler is used in an amount of 0.05 to 20 parts by weight, based on one part by weight of the air purifier.

8. The polymer composition according to claim 5, wherein the flame retardant is used in an amount of 0.01 to 10 parts by weight, based on one part by weight of the air purifier.
